(19) United States
(12) Patent Application Publication
(10) Pub. No.: US 2016/0053217 A1
(43) Pub. Date: Feb. 25, 2016

(54) AUTOMATIC STIMULATOR FOR CELL CULTURES BASED ON VIBRATIONS

(71) Applicants: Jorge Arturo REY CUBILLOS, Bogota (CO); Astrid RUBIANO FONSECA, Bogota (CO); UNIVERSIDAD MILITAR NUEVA GRANADA, Bogota (CO)

(72) Inventors: Jorge Arturo REY CUBILLOS, Bogota (CO); Astrid RUBIANO FONSECA, Bogota (CO)

(21) Appl. No.: 14/779,242

(22) PCT Filed: Mar. 14, 2014
(86) PCT No.: PCT/IB2014/059844

§ 371 Date: Sep. 22, 2015

(30) Foreign Application Priority Data
Mar. 22, 2013 (CO) 13-057430

(51) Int. Cl. C12M 1/42; C12M 1/24

(52) U.S. Cl. CPC C12M 35/04; C12M 23/08

(57) ABSTRACT

The present invention relates to an automatic stimulator based on vibrations for cell cultures, fulfilling the function of activating the proliferation of osteoprogenitor cells and of activating the expression of the genes involved in the process of cell differentiation toward the osteogenic line. This effect is produced in the cells by the application of mechanical vibrational load. In this respect, the operation of the stimulator of the present invention comprises a system incorporating an actuator of mechanical type, controlled and regulated according to the specific requirements defined by the user. The actuator is the source of the vibrations which will be applied upon the flasks containing the cell cultures. The automatic stimulator for cell cultures is characterized by being controllable and programmable from a computer through the storage of routines of stimulation and repose, with programming of intensity of the load graduated in G (gravitational) units, being repeated in an automatic manner a number of times determined by the user, it being appropriate for performing routines in the laboratory.

### FIELD OF TECHNOLOGY

The present invention relates to an automatic or automated stimulator based on vibrations for cell cultures, fulfilling the function of activating the proliferation of osteoprogenitor cells and of activating the expression of the genes involved in the process of cell differentiation toward the osteogenic line. This effect is produced in the cells by the application of mechanical vibrational load.

In this respect, the operation of the stimulator of the present invention comprises a system incorporating an actuator of mechanical type, controlled and regulated according to the specific requirements defined by the user. The actuator is the source of the vibrations which will be applied upon the flasks containing the cell cultures.

The automated stimulator for cell cultures is characterized by being controllable and programmable from a computer through the storage of routines of stimulation and repose, with programming of intensity of the load graduated in G (gravitational) units, being repeated in an automatic manner a number of times determined by the user, it being appropriate for performing routines in the laboratory.

### BACKGROUND OF THE INVENTION

In the state of the art there exist very few systems to carry out a stimulation of cell cultures and they are principally based on a mechanical stimulation of the cells by vibrations. The few devices in existence have fairly complex and expensive systems, not being appropriate for carrying out routine tests in the laboratory.

In this sense, the document JP 2003021634 relates to a method and program for the analysis of variation in gene expression utilized to obtain information appropriate in respect of the significance of the vibration of the intensity of expression, whilst robustness is ensured by means of samples whereupon the level is determined in accordance with the intensity of expression of the gene.

A disadvantage presented by the invention of said document is that it does not make use of a device, being solely a logical procedure.

In addition, the document JP 2003042952 divulges an infrared molecular vibration analyzer, a system to measure the data of the organism, and a method for measuring said data, wherein an infrared absorption spectrum is detected solely on placing an oral cavity mucous membrane in contact with a probe. The spectrum detected is processed, including the information of peaks and the data of the organisms, considering at least one lipid and one sugar; in this manner the expression of the gene may be obtained in an easy and non-invasive manner by analyzing the data from neural networks.

Another document in the state of the art in respect of this type of technology is the Japanese patent JP 2003320038 relating to the development of nuclear magnetic resonance therapy to induce the expression of genes by means of the vibration of a charged particle, utilizing the magnetic field and the weak electric current generated in this same manner, wherein the frequency of vibration of a base in an annular dimension is defined in a quantum mechanical manner, wherein the defined frequency of vibration of the base is correlated with a flip angle of the charged particle.

A disadvantage presented by the invention of said document is that it solely makes reference to a process and a series of algorithms establishing the parameters required to develop a magnetic resonance therapy, not promoting cell proliferation nor the expansion of the cell cultures.

In addition to the foregoing documents, there exists the patent application CO 10-164988 relating to a mechatronic apparatus to carry out the mechanical stimulation (tension and compression) of cell cultures in vitro, transmitting different intensities of force to the cells adhered to the surface in culture flasks, having the objective of studying and analyzing the different phases of the cell cycle, wherein the mechanical stimulation is based on the production of cyclical deformations of the cells at different magnitudes and frequencies in the culture flasks, rendering the possibility of regulating the periods of stimulation and repose by means of an automated control system permitting programming of the number of cycles of activation or repose to be realized in a period established by the user, wherein it is desired to carry out the mechanical stimulation on each of the culture flasks. The control system is responsible for sending different signals to the servomotor which realizes rotations to transmit the movement to a camshaft producing the mechanical stimulation of the culture flasks.

Nevertheless, there exists a series of differences, principally in the functioning of the device, by virtue of the fact that said application is based upon the use of a piston creating a compression and tension of the cell in the vessel, whilst the present invention is based upon the movement of the flask in the 3 axes, at different frequencies and intensities, in order to thus create a vibration stimulating the cells without deforming them. Moreover, said patent uses a single motor having a shaft transmitting the movement and, in this manner, moving the pistons deforming the flasks; on the contrary in the present invention the stimulation is undertaken in an independent manner upon each flask wherein the cells to be stimulated are located.

Considering the foregoing information, it is evident that the need existed in the state of the art to design and implement a mechatronic apparatus permitting carrying out of the stimulation of cell cultures by means of vibrations, wherein the cell culture may be easily located in a culture flask or container to produce the molecular excitation required to activate the genes regenerating the human osseous tissue.

In addition to the advantages of adaptation and handling, the device continuously monitors the magnitude and frequency of the vibrational wave produced in the three axes (x, y, z). This monitoring is carried out through the direct contact of the electronic sensor with the flask, consequently minimizing the possibility of error in the magnitude and frequency of the stimulant wave applied to the cell, in such manner that said wave activates the genes involved in the formation of the osseous tissue. Moreover, it is required that the apparatus be easy to use and present great simplicity, having the objective of reducing costs of operation and maintenance of the same.

### DESCRIPTION OF THE FIGURES

The invention may be better understood by means of the figures wherein there is shown each of the elements which make up the automatic stimulator for cell cultures based on vibrations which it is desired to protect. Furthermore, the figures show the reference numbers assigned to the elements forming said apparatus.

FIG. 1 corresponds to the general layout of the automatic stimulator apparatus for cell cultures based on vibrations of the present invention, composed of the control device (1), the actuator device (2), and the communication device (3).

FIG. 2 corresponds to a detailed view of the control device (1) of the present invention.

FIG. 3 corresponds to a detailed view of the actuator device (2) of the present invention.

FIG. 3A corresponds to a lateral view of the actuator device (2) of the present invention.

FIG. 3B corresponds to a detailed view of solely the elements 22 (movement isolating material) and fins 221 of the actuator device (2) of the present invention.

FIG. 3C corresponds to a detailed view of solely the elements 23, 231, the hinge 26 and the element 27 of the actuator device (2) of the present invention.

### DETAILED DESCRIPTION OF THE INVENTION

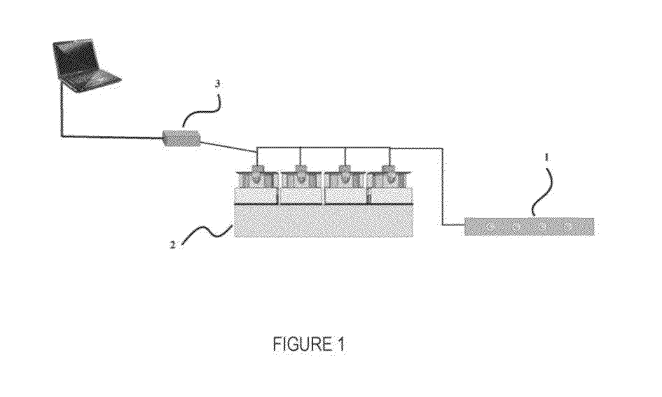

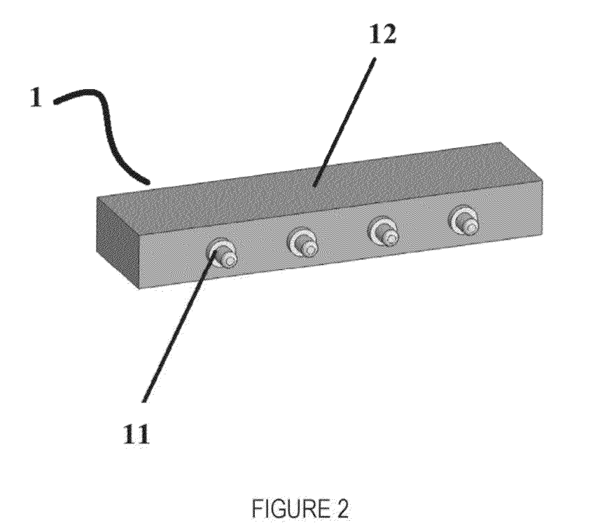

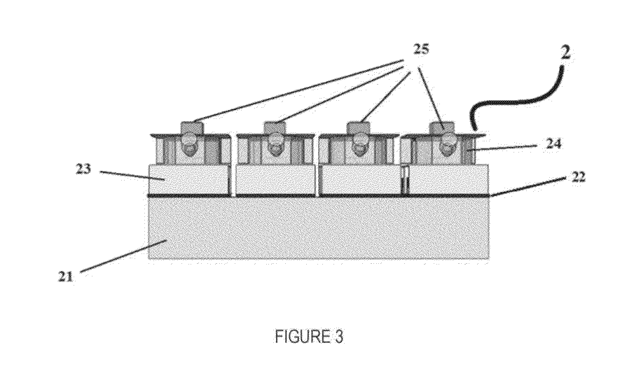

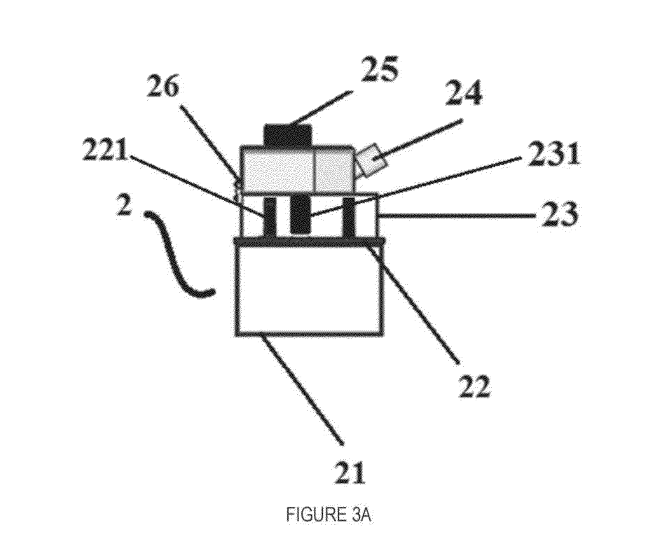

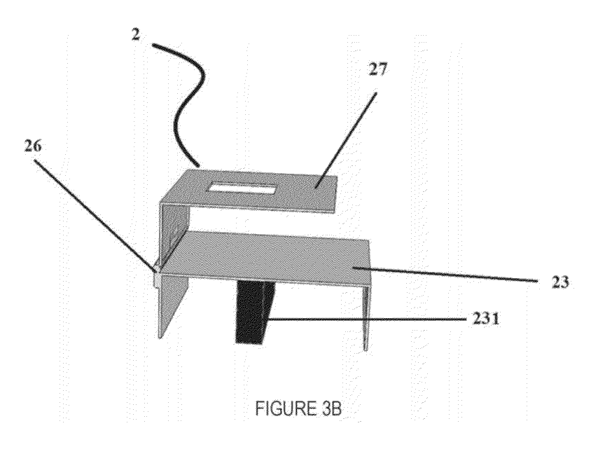

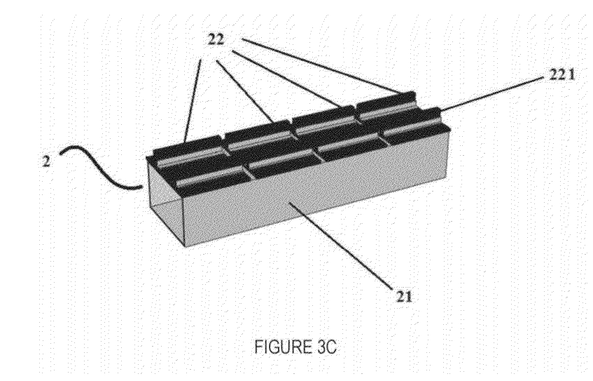

The object of the present invention is to provide an automatic stimulator apparatus for cell cultures based on vibrations, basically comprising:
A control device (1), an actuator device (2) and a communication device (3), as shown in FIG. 1.
The control device (1) is composed of four knobs 11, being potentiometers responsible for regulating the current of the motor in each sensing and actuation device 25 together with an integrated circuit, contained in casing 12, as shown in FIG. 2.
The actuator device (2) is composed of a base 21, a covering of movement isolating material 22, four supports for cell culture flasks 23, flasks 24, sensing and actuation devices 25, as shown in FIG. 3A.

The base 21 is covered by the movement isolating material 22 to isolate the vibrations applied from one flask to another and, consequently, to independently stimulate cells contained in each of the flasks 24.

As illustrated in FIG. 3B, the isolating covering 22 has fins 221 projecting from the base thereof and reaching as far as the lower part of the support 23 which in turn has adhered thereto one or several fins 231 of the same material of the covering 22 and which reaches as far as the base of the isolating covering 22. This configuration permits that each system be independent of the others and renders that the different vibrations of the systems are not modified or altered by the conditions of vibration of the neighboring systems.

The cell culture flasks 24 are borne on the supports 23 and 27 connected by the hinge 26, as shown in FIG. 3A, permitting the simple location of the flask and also placing in an appropriate and precise manner the sensing and actuation device 25 on each thereof upon the element 27 having a perforation in the upper part thereof. In the same manner, the support 23 connected by the hinge 26 to the support 27 permits adjusting the flasks such that they are not displaced in any direction whilst the cells are stimulated by the sensing and actuation device 25.

The sensing and actuation device 25 is composed of an accelerometer and a motor. The accelerometer is responsible for measuring the vibrations (in terms of acceleration, velocity and displacement) produced in each of the axes x, y, z, wherefrom there is determined the intensity of stimulation of the vibrations produced by the motor on each of the flasks 24.
The signals delivered by the device 25 are processed in the communication device (3), having the function of transmitting in digital form the magnitudes of the vibrations produced in the axes x, y, z of each of the cell culture flasks 24.
The communication device (3) sends by USB communication to a PC the information in relation to the magnitudes of the vibrations in the three axes applied upon each of the flasks 24.

The principle of operation of the automatic stimulator apparatus for cell cultures based on vibrations according to the present invention comprises the generation of vibrations by the sensing and actuation device 25, these vibrations being produced by the motor contained in said device 25 and the regulation of the intensity of these vibrations being realized by the control device (1).

Subsequently, the vibrations are sensed by an accelerometer contained in the sensing and actuation device 25, the digital information delivered by the accelerometer is processed and sent to the PC by the communication device (3), this permitting continual monitoring of the magnitude and frequency of the stimulation by means of vibrations transferred to the flask 24 and these, in turn, transfer these vibrations to the cell cultures contained therein.

In one embodiment of the invention, each flask 24 has a support 23 and a sensing and actuation device 25 contained in the actuator device (2), a communication device (3), and a control device (1).

In another embodiment of the present invention, the supports 23 and the base 21 of the actuator device (2) are ideally fabricated in a metallic material made for example from, inter alia, aluminum, copper and stainless steel, zinc, brass, bronze, nickel and silver, or from any metal having high hardness providing required stability to the structure. In general a material may be utilized the Young's modulus whereof lies between 8×10¹⁰ N/m² and 20×10¹⁰ N/m².

Additionally, the movement isolating material for the covering 22 of FIG. 3 must be a material permitting isolating in an independent manner of the vibrational waves experienced by each flask; examples of suitable materials for this function are, inter alia, elastomer materials such as rubber, natural rubber, polybutadiene, neoprene and silicone. In general a material may be utilized the Young's modulus whereof lies between 2×10⁶ N/m² and 3 N/m².

In other embodiments of the invention each flask may receive different magnitudes and frequencies in terms of the vibrational waves. Moreover, the device of the invention is versatile by virtue of the fact that it may have different cultures in each flask and there may be more than four culture flasks per device, depending on the size of the incubators wherein the experiments are realized.

1. Automatic stimulator based on vibrations for cell cultures, comprising:
a) a control device,
b) an actuator device and
c) a communication device,
wherein the control device is composed of four knobs 11 having an integrated circuit contained within a casing; the actuator device is composed of a base, a covering of movement isolating material, a support for cell culture flask, a flask, and a sensing and actuation device; the communication device sending by USB communication to a PC the information relating to the magnitudes of the vibrations in the three axes applied upon each of the flasks.

2. Automatic stimulator based on vibrations according to claim 1, wherein the base in the actuator device is covered by a movement isolating material.

3. Automatic stimulator based on vibrations according to claim 2, wherein the movement isolating material on the base is an elastomer material.

4. Automatic stimulator based on vibrations according to claim 3, wherein the movement isolating material on the base is an elastomer material selected from rubber, natural rubber, polybutadiene, neoprene and silicone.

5. Automatic stimulator based on vibrations according to claim 1, wherein the supports 21, 23 and 27 of the actuator device are of a metal selected from aluminum, copper and stainless steel, zinc, brass, bronze, nickel and silver.

6. Automatic stimulator based on vibrations according to claim 1, wherein the control device regulates the intensity of the vibration of the sensing and actuation device of the actuator device.

7. Automatic stimulator based on vibrations according to claim 1, wherein on the actuator device the covering of movement isolating material has fins projecting from the base thereof and reaching as far as the lower part of the support, which in turn has adhered thereto a fin of the same material of the covering and which reaches as far as the base of the isolating covering.
